(19) United States
(12) Patent Application Publication
(10) Pub. No.: US 2023/0331674 A1
(43) Pub. Date: Oct. 19, 2023

(54) N-((1-BENZYLPIPERIDIN-3-YL)METHYL)-N-(2-METHOXYETHYL)NAPHTHALENE-2-SULFONAMIDE FOR THE TREATMENT OF CANINE COGNITIVE DYSFUNCTION AND OTHER FORMS OF DEMENTIA IN DOGS

(71) Applicant: UNIVERZA V LJUBLJANI, Ljubljana (SI)

(72) Inventors: Maja ZakoSek, Velenje (SI); Sonja Prpar Mihevc, Ljubljana (SI); Gregor Majdic, Ljubljana (SI); Urban Ko{hacek over (s)}ak, Ljubljana (SI); Stanislav Gobec, Slovenia (SI)

(21) Appl. No.: 18/027,144

(22) PCT Filed: Sep. 21, 2021
(86) PCT No.: PCT/EP2021/075883

§ 371 Date: Mar. 20, 2023

(30) Foreign Application Priority Data
Sep. 22, 2020 (LU) LU102073

(51) Int. Cl. C07D 211/28; A61P 25/28

(52) U.S. Cl. CPC C07D 211/28; A61P 25/28

(57) ABSTRACT

The present invention relates to the fields of medicinal chemistry and veterinary medicine, and in particular to N-((1-benzylpiperidin-3-yl)methyl)-N-(2-methoxyethyl)naphthalene-2-sulfonamide as a pharmaceutically active compound. The compound of the present invention has been proven for the first time to be particularly useful for the treatment of canine cognitive dysfunction and other forms of dementia in dogs which may be therapeutically modified by altering the activity of butyrylcholinesterase.

### TECHNICAL FIELD

The present invention relates to the fields of medicinal chemistry and veterinary medicine, and in particular to N-((1-benzylpiperidin-3-yl)methyl)-N-(2-methoxyethyl)naphthalene-2-sulfonamide as a pharmaceutically active compound. The compound of the present invention has been proven for the first time to be particularly useful for the treatment of canine cognitive dysfunction and other forms of dementia in dogs which may be therapeutically modified by altering the activity of butyrylcholinesterase.

### BACKGROUND ART

Neurodegenerative diseases represent serious and increasing health problems in people and also in dogs and other animals. Alzheimer's disease (AD)—associated decline of the neurological and cognitive function is one of the most serious neurodegenerative diseases and numbers of affected individuals are raising exponentially worldwide (GBD 2016 Dementia Collaborators Lancet Neurol. 2019, 18, 88-106). The disease now affects nearly 50 million people, and the incidence is predicted to triple by 2050. Similar disease in dogs, called canine cognitive dysfunction, (CCD) is affecting more than 30 million dogs in the USA and over 15 million dogs in Europe (Bosch M. N. et al. Curr. Alzheimer Res. 2012, 9, 298-314). CCD affects up to 60% of older dogs, mostly dogs older than 11 years (Fast R. et al. J. Vet. Intern. Med. 2013, 27, 822-829) and age is the most prominent risk factor for the development of this disease (Katina S. et al. Acta Vet. Scand. 2016, 58, 17; Salvin H. E. et al. Vet. J. 2011, 188, 331-336).

CCD, also referred to as cognitive dysfunction syndrome (CDS), has many similarities with AD in humans (Rofina J. E. et al. Brain Res. 2006, 1069, 216-226). Patients with AD or CCD show similar neuropathological changes affecting cerebral gyri (cerebral atrophy), cerebral angiopathy, and ventricular enlargement (Pan Y. et al. Front. Nutr. 2018, 5, 127). Cerebral atrophy is a consequence of neuronal death occurring throughout the brain and leads to memory loss. When these diseases progress people and animals can lose their ability to move, communicate, and show diminished cognitive abilities (Holmes C. and Amin J. Psychiatric Disorders 2016, 44, 687-690). Clinically CCD in dogs includes a number of behavioral alterations such as changes in sleep-wake pattern, decreased social interaction, elimination habits, lack of activity, and disorientation, as well as increasing anxiety (Landsberg G. M. et al. Vet. Clin. North Am. Small. Anim. Pract. 2012, 42, 749-768). Deficits in learning and memory have also been documented (Landsberg G. M. et al. Vet. Clin. North Am. Small. Anim. Pract. 2012, 42, 749-768). In the early stages, these signs are often attributed to normal aging and because of the lack of knowledge about CCD among owners and veterinarians, it is often underdiagnosed (Benzal A. S. and Rodriguez A. G. Pet Behav. Sci. 2016, 1, 47-59).

Diagnosis of CCD is based on the recognition of behavioral signs and exclusion of other medical causes that might mimic CCD or complicate its diagnosis. Drugs, diets, and supplements are available that might slow the disease progression by various mechanisms including reducing oxidative stress and inflammation or improving mitochondrial and neuronal function (Pan Y. et al. Front. Nutr. 2018, 5, 127). However, the disease cannot be cured and there are no effective treatments for CCD and AD at the moment. Due to the high prevalence of age-related dementias in people and dogs, it is imperative to find a cure for these devastating diseases.

The neuropathological changes, potentially leading to dementia, have been observed in the brains of AD and CCD patients, these include extracellular amyloid plaques, intracellular neurofibrillary tangles, oxidative brain damage, neuroinflammation, and others (Masters C. L. et al. Nat. Rev. Dis. Primers 2015, 1, 15056; Prpar Mihevc S. and Majdič G. Front. Neurosci. 2019, 13, 604). Cholinergic dysfunction is prominent in AD and similarly, the activity of the canine cholinergic system declines with age (Araujo M. G. and Lindhe J. et al. J. Clin. Periodontol. 2005, 32, 212-218; Contestabile A. Behav. Brain Res. 2011, 221, 334-340). Current treatment options for AD are only symptomatic and three out of four available drugs are acetylcholinesterase inhibitors (Winblad B. et al. Lancet Neurol. 2016, 15, 455-532). Current pharmacological interventions are essential, since they temporarily improve cognition (Dou K. X. et al. Alzheimers Res. Ther. 2018, 10, 126), although cannot stop the progression of AD. Unfortunately, there have been more than 2000 AD failed clinical trials (Liu P. P. et al. Signal Transduct. Target Ther. 2019, 4, 29), nevertheless there are more than 200 currently ongoing. Some of these drugs have been tested in dogs, and often have similar effects as reported for AD patients (Araujo J. A. et al. J. Alzheimers Dis. 2011, 26, 143-155; Neumann U. et al. EMBO Mol. Med. 2018, 10, e9316; May P. C. et al. J. Neurosci. 2015, 35, 1199-1210; Studzinski C. M. Prog. Neuropsychopharmacol. Biol. Psychiatry 2005, 29, 489-498).

Similarly to human AD, cholinergic hypofunction is present in dogs with CCD (Araujo J. A. et al. J. Alzheimers Dis. 2011, 26, 143-155), and cholinergic system is therefore an interesting target for the symptomatic treatment of CCD. Such treatments could work through the modulation of acetylcholinesterases (AChEs) and butyrylcholinesterases (BChE), which are all an important part of the cholinergic system.

### SUMMARY OF INVENTION

The invention relates to an inhibitor of the enzyme butyrylcholinesterase with the formula I.

Particularly, the invention relates to the compound of formula (I), optionally in the form of a stereoisomer, such as enantiomer, or a mixture of at least two stereoisomers, such as at least two enantiomers, or a pharmaceutically acceptable salt, hydrate or solvate thereof, for use in the treatment of canine cognitive dysfunction and other forms of dementia in dogs.

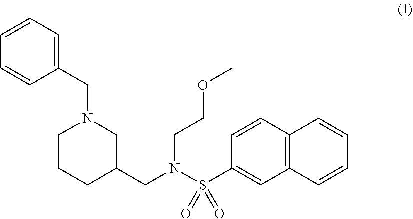 [SMILES: COCCN(CC1CCCN(Cc2ccccc2)C1)S(=O)(=O)c1ccc2ccccc2c1]

As demonstrated in the Examples, the compound of the formula (I) has shown unexpected positive effects in dogs suffering from canine cognitive dysfunction. This is the first proven use of the compound of formula (I) in the treatment of a diagnosed disease in clinical setting, making the compound an excellent drug for the treatment of canine cognitive dysfunction and other forms of dementia in dogs.

### Technical Problem

Currently, there is only one registered drug for treatment of canine cognitive dysfunction in US and none in Europe. Since this drug has limited efficacy, there is a need for the discovery of novel medications for the treatment of canine cognitive dysfunction and other dementias in dog.

### Solution to Problem

The above problem is solved by the present inventors based on the surprising finding that the compound of formula (I)

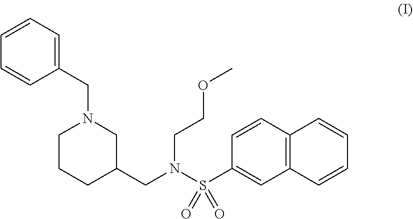 [SMILES: COCCN(CC1CCCN(Cc2ccccc2)C1)S(=O)(=O)c1ccc2ccccc2c1]

shows excellent efficacy in dogs suffering from canine cognitive dysfunction.

The present invention thus provides in a first aspect a compound of (I)

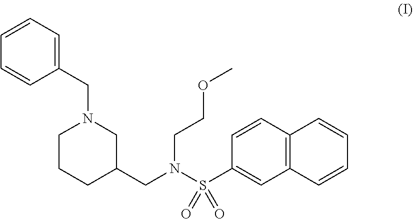 [SMILES: COCCN(CC1CCCN(Cc2ccccc2)C1)S(=O)(=O)c1ccc2ccccc2c1]

optionally in the form of a stereoisomer, such as enantiomer, or a mixture of at least two stereoisomers, such as at least two enantiomers, or a pharmaceutically acceptable salt, hydrate or solvate thereof, for use in the treatment of canine cognitive dysfunction or other form of dementia in dog.

The invention further relates to the use of the compound of formula I, optionally in the form of a stereoisomer, such as enantiomer, or a mixture of at least two stereoisomers, such as at least two enantiomers, or a pharmaceutically acceptable salt, hydrate or solvate thereof, as the active ingredient for the preparation of a medicament for the treatment of canine cognitive dysfunction or other form of dementia in dog.

The compound of formula I is an inhibitor of the enzyme butyrylcholinesterase and is used for the treatment of symptoms of canine cognitive dysfunction, as well as all forms of dementias and cognitive disorders in dogs related to decreased cholinergic neurotransmission.

The invention further relates to a pharmaceutical composition for use in the treatment of canine cognitive dysfunction or other form of dementia in dog, comprising a therapeutically effective amount of the compound of formula (I), optionally in the form of a stereoisomer, such as enantiomer, or a mixture of at least two stereoisomers, such as at least two enantiomers, or a pharmaceutically acceptable salt, hydrate or solvate thereof.

The invention is related to parenteral, per oral or other pharmaceutically acceptable forms containing the compound with the formula I.

Beside active pharmaceutical ingredient, the pharmaceutical composition of the invention can contain one or more excipients suitable for the intended route of administration.

Pharmaceutical compositions are prepared using standard procedures.

Pharmaceutical compositions can be prepared in the way that ensures the sustained release of the active pharmaceutical ingredient.

The invention further relates to a method of treating canine cognitive dysfunction or other form of dementia in a dog in need of treatment, comprising administering an effective amount of the compound of formula (I), optionally in the form of a stereoisomer, such as enantiomer, or a mixture of at least two stereoisomers, such as at least two enantiomers, or a pharmaceutically acceptable salt, hydrate or solvate thereof, to said dog.

The dose, frequency and way of use are dependent from several factors, which are further dependent also from the active pharmaceutical ingredient used, its pharmacokinetic properties and patient's condition.

The compound of formula I can be prepared using modified synthetic procedure described in the literature (Košak U. et al. Tetrahedron Lett. 2014, 55, 2037-2039; Košak U. et al. Sci. Rep. 2016, 6, 39495).

Having generally described this invention, a further understanding can be obtained by reference to the following examples, which are provided herein for purposes of illustration only, and are not intended to be limiting unless otherwise specified.

### EXAMPLES

### Example 1: Synthesis of (±)-N-((1-benzylpiperidin-3-yl)methyl)-N-(2-methoxyethyl)naphthalene-2-sulfonamide

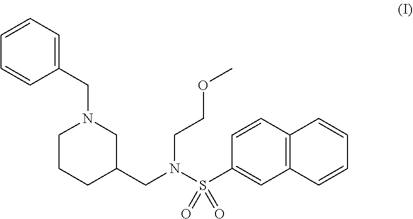 [SMILES: COCCN(CC1CCCN(Cc2ccccc2)C1)S(=O)(=O)c1ccc2ccccc2c1]

### STEP 1: Synthesis of (±)-1-benzoylpiperidine-3-carboxylic Acid

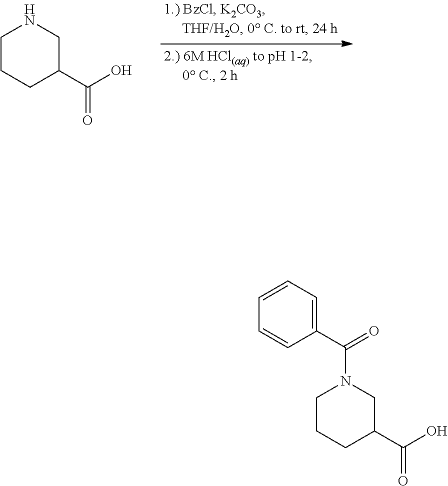 [SMILES: O=C(O)C1CCCNC1 ; O=C(O)C1CCCN(C(=O)c2ccccc2)C1]

To a 4-L round-bottomed flask equipped with a stirring bar, piperidine-3-carboxylic acid (100 g, 0.774 mol, 1.0 equiv) was added. THF (600 mL) was added and the resulting suspension was stirred. H₂O (800 mL) was added, and after all piperidine-3-carboxylic acid dissolved, the solution was cooled to 0° C. K₂CO₃ (536 g, 3.878 mol, 5.0 equiv) were added portion-wise. A solution of benzoyl chloride (90 mL, 0.774 mol, 1.0 equiv) in THF (200 mL) was then added drop-wise. The reaction mixture was allowed to warm to room temperature, stirred for 24 hours, transferred into a 2-L separating funnel and washed with EtOAc (3×1 L). The aqueous phase was transferred into a 5-L beaker equipped with a stirring bar, stirred, cooled to 0° C., and adjusted to pH 1-2 with 6 M aqueous HCl solution. A white solid precipitated and the suspension was stirred at 0° C. for 2 hours. The white precipitate was then collected in a Büchner funnel under suction filtration, washed with H₂O (3×500 mL) and dried in a drying oven at 80° C. to constant mass to produce 169 g of (±)-1-benzoylpiperidine-3-carboxylic acid. This product was used in the next step without further purification.

Product appearance: white solid

Yield: 93%

Melting point: 171-175° C.

TLC: 0.53 (MeCN-MeOH—H₂O=3/1/1, v/v/v)

IR (ATR): 2865, 2563, 1709, 1584, 1564, 1464, 1277, 1212, 929, 861, 791, 729, 632, 572 cm⁻¹.

¹H NMR (400 MHz, DMSO-d6): δ=1.45-1.69 (3H, m), 1.96-2.00 (1H, m), 2.42-2.46 (1H, m), 3.00-3.17 (2H, m), 3.45-3.63 (1H, m), 4.13-4.43 (1H, m), 7.37-7.52 (5H, m), 12.44 (1 H, bs).

¹³C NMR (100 MHz, DMSO-d6): δ=23.63, 24.40, 26.79, 40.63, 41.54, 43.41, 47.29, 48.72, 48.72, 126.64, 128.33, 129.32, 136.24, 169.14, 174.24

HRMS (ESI⁺): m/z calculated for C₁₃H₁₆NO₃: 234.1130; found: 231.1129.

CHN analysis: calculated for C₁₃H₁₅NO₃: C, 66.94; H, 6.48; N, 6.00. Found: C, 67.15; H, 6.74; 6.16.

### STEP 2: Synthesis of (±)-1-benzoyl-N-(2-methoxyethyl)piperidine-3-carboxamide

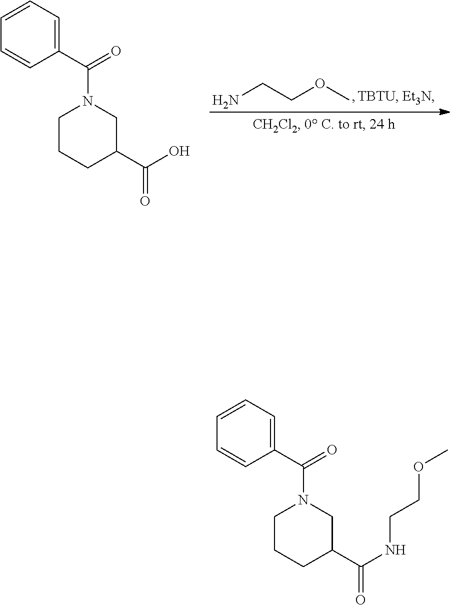

To a 4-L round-bottomed flask equipped with a stirring bar, 1-benzoylpiperidine-3-carboxylic acid (169 g, 0.725 mol, 1.0 equiv) was added followed by CH₂Cl₂ (3 L). The resulting suspension was stirred and cooled to 0° C. Et₃N (202 mL, 1.449 mol, 2.0 equiv) was added drop-wise. After all the solid dissolved, O-(benzotriazol-1-yl)-N,N,N′,N′-tetramethyluronium tetrafluoroborate (TBTU) (233 g, 0.725 mol, 1.0 equiv) was added in two equal portions. After 1 hour, 2-methoxyethylamine (125 mL, 1.449 mol, 2.0 equiv) was added drop-wise via a dropping funnel. The reaction mixture was allowed to warm to room temperature, stirred for 24 h and then divided up into 3 portions of approximately 1 L. Every portion was transferred into a 2-L separating funnel, washed with H₂O (2×1 L), 0.5 M aqueous HCl solution (2×1 L) followed by saturated aqueous NaHCO₃ solution (2×1 L), and dried over anhydrous Na₂SO₄. All dried organic phases were pooled and evaporated, to produce 186 g of (±)-1-benzoyl-N-(2-methoxyethyl)piperidine-3-carboxamide as a colourless oil. This product was used in the next step without further purification

Product appearance: colourless oil

Yield: 86%

HRMS (ESI+): HRMS (ESI⁺): m/z calculated for C₁₆H₂₃N₂O₃: 291.1709; found: 291.1707.

### STEP 3: Synthesis of (±)-N-((1-benzylpiperidin-3-yl)methyl)-2-methoxyethan-1-amine

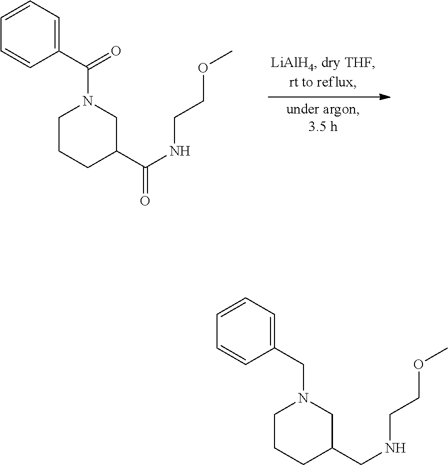 [SMILES: COCCNC(=O)C1CCCN(C(=O)c2ccccc2)C1 ; COCCNCC1CCCN(Cc2ccccc2)C1]

To a 1-L tree-neck round-bottomed flask equipped with a stirring bar and a reflux condenser, LiAlH₄ (14.200 g, 0.374 mol, 3.5 equiv) was added under an argon atmosphere. Dry THF (ca. 450 mL) was added with a double-tipped needle. A solution of (±)-1-benzoyl-N-(2-methoxyethyl)piperidine-3-carboxamide (31 g, 0.107 mol, 1.0 equiv) in dry THF (ca. 150 mL) was added with a double-tipped needle, and the reaction mixture was refluxed for 3.5 hours. The mixture was then cooled to 0° C. and the excess hydride was decomposed by drop-wise addition of H2O (14.2 mL) followed by 15% aqueous NaOH solution (14.2 mL) and then H2O (42.6 mL). The suspension was allowed to warm to room temperature, stirred for 12 h then filtered under suction. The white precipitate was washed thoroughly with THF (5×200 mL). This reaction was performed in the same way 5 more times to use up all of compound (±)-1-benzoyl-N-(2-methoxyethyl)piperidine-3-carboxamide. Filtrates of all 6 reactions were pooled together and evaporated to produce 156 g of (±)-N-((1-benzylpiperidin-3-yl)methyl)-2-methoxyethan-1-amine as a slightly golden liquid (93% yield). This product was used in the next step without further purification.

Product appearance: slightly golden liquid

Yield: 93%

HRMS (ESI+): m/z calculated for C₁₆H₂₇N₂O: 263.2123; found: 263.2128.

### STEP 4: Synthesis of (±)-N-((1-benzylpiperidin-3-yl)methyl)-N-(2-methoxyethyl)naphthalene-2-sulfonamide

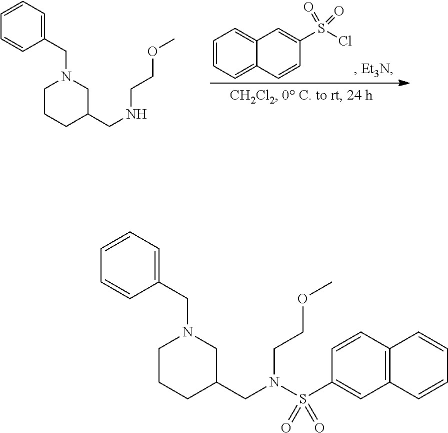

To a 2-L round-bottomed flask containing (±)-N-((1-benzylpiperidin-3-yl)methyl)-2-methoxyethan-1-amine (156 g, 0.595 mol, 1.0 equiv), CH₂Cl₂ (1.5 L) was added. A stirring bar was added to the resulting solution, which was then stirred and cooled to 0° C. Et₃N (83 mL, 0.595 mol, 1.0 equiv) was added drop-wise. After 30 minutes, naphthalene-2-sulfonyl chloride (135 g, 0.595 mol, 1.0 equiv.) was added portion-wise. The reaction mixture was allowed to warm up to room temperature, stirred for 24 hours and divided up into 2 portions of approximately 1 L. Every portion was transferred into a 2-L separating funnel, washed with H₂O (1 L), followed by 1 M aqueous NaOH solution (1 L), and dried over anhydrous Na₂SO₄. Both dried organic phases were pooled together and evaporated, to produce 256 g of (±)-N-((1-benzylpiperidin-3-yl)methyl)-N-(2-methoxyethyl)naphthalene-2-sulfonamide as a slightly golden oil. An analytically pure sample was obtained by purifying via flash column chromatography using CH₂Cl₂-MeOH (30:1, v/v) as the eluent. The rest of crude (±)-N-((1-benzylpiperidin-3-yl)methyl)-N-(2-methoxyethyl)naphthalene-2-sulfonamide was used in the next step without further purification.

Product appearance (analytically pure sample): colorless oil

Yield: 95%

TLC: R_{f}=0.57 (CH₂Cl₂-MeOH=10:1, v/v)

IR (ATR): 2928, 2803, 1452, 1333, 1154, 1115, 1072, 983, 883, 859, 817, 732, 699, 650, 615 cm⁻¹

¹H NMR (400 MHz, CDCl₃): δ=0.95-1.04 (1H, m), 1.54 (1 H, bs), 1.66-1.74 (3H, m), 1.98 (2 H, bs), 2.76 (2H, d, J=30.4 Hz), 3.06-3.16 (2H, m), 3.21 (3 H, s), 3.30 (2H, t, J=6.3 Hz), 3.45-3.48 (4H, m), 7.23-7.30 (5H, m), 7.59-7.66 (2H, m), 7.76 (1 H, dd, J1=8.6 Hz, J2=1.8 Hz), 7.89-7.97 (3H, m), 8.38 (1H, d, J=1.4 Hz).

¹³C NMR (100 MHz, CDCl₃): δ=24.54, 28.21, 34.82, 48.04, 53.21, 53.89, 57.60, 58.66, 63.44, 71.10, 122.52, 126.85, 127.39, 127.78, 128.05, 128.36, 128.56, 129.07, 129.11, 129.14, 132.07, 134.59, 136.40, 138.25.

HRMS (ESI+): m/z calculated for C₂₆H₃₃N₂O₃S 453.2212; found 453.2209.

### Example 2: Synthesis of (±)-N-((1-Benzylpiperidin-3-Yl)Methyl)-N-(2-Methoxyethyl)Naphthalene-2-Sulfonamide Hydrochloride

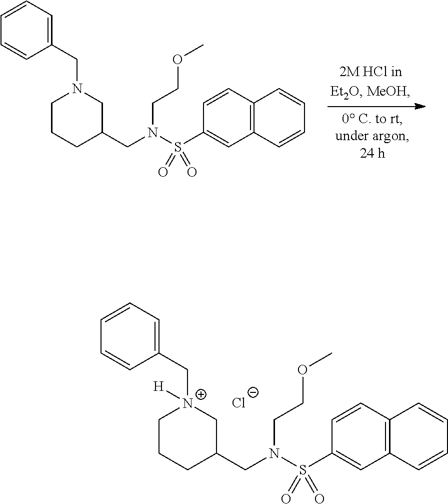 [SMILES: COCCN(CC1CCCN(Cc2ccccc2)C1)S(=O)(=O)c1ccc2ccccc2c1 ; COCCN(CC1CCC[NH+](Cc2ccccc2)C1)S(=O)(=O)c1ccc2ccccc2c1 ; [Cl-]]

To a 2-L round-bottomed flask containing (±)-N-((1-benzylpiperidin-3-yl)methyl)-N-(2-methoxyethyl)naphthalene-2-sulfonamide (128 g, 0.282 mol, 1.0 equiv), MeOH (640 mL) was added. A stirring bar was added to the resulting solution, which was then stirred, agitated with a stream of argon for 30 min, and cooled to 0° C. 2 M HCl solution in Et₂O (156 mL, 0.310 mol, 1.1 equiv) was added with a double-tipped needle. The reaction mixture was allowed to warm up to room temperature, stirred for 24 hours and evaporated. MeOH (240 mL) was added to the residue, followed by a stirring bar. The solution was stirred and Et₂O (1.5 L) was added slowly. A white solid precipitated and the suspension was stirred at room temperature for 3 hours. The white precipitate was then collected in a Buchner funnel under suction filtration and washed with Et₂O (2×500 mL). This reaction was performed in the same way with the rest of (±)-N-((1-benzylpiperidin-3-yl)methyl)-N-(2-methoxyethyl)naphthalene-2-sulfonamide (128 g). The white solid from both batches were pooled together and dried in a desiccator in vacuo at room temperature in the presence of crushed NaOH to constant mass to produce 192 g of N-((1-benzylpiperidin-3-yl)methyl)-N-(2-methoxyethyl)naphthalene-2-sulfonamide hydrochloride. The mother liquids from both crystallizations were pooled together and evaporated to produce 84 g of impure (±)-N-((1-benzylpiperidin-3-yl)methyl)-N-(2-methoxyethyl)naphthalene-2-sulfonamide hydrochloride as a slightly golden oil.

Product appearance: white solid

Yield: 69%

### Example 3: Purification of Impure (±)-n-((1-benzylpiperidin-3-yl)methyl)-n-(2-methoxyethyl)naphthalene-2-sulfonamide Hydrochloride

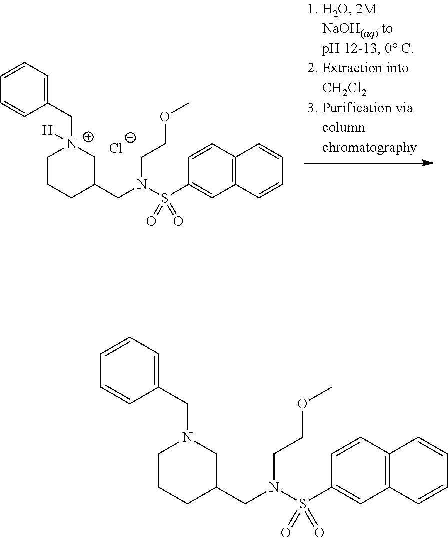 [SMILES: COCCN(CC1CCC[NH+](Cc2ccccc2)C1)S(=O)(=O)c1ccc2ccccc2c1 ; [Cl-] ; COCCN(CC1CCCN(Cc2ccccc2)C1)S(=O)(=O)c1ccc2ccccc2c1]

To a 2-L round-bottomed flask containing impure (±)-N-((1-benzylpiperidin-3-yl)methyl)-N-(2-methoxyethyl)naphthalene-2-sulfonamide hydrochloride (84 g), H₂O (840 mL) was added. A stirring bar was added to the mixture which was then stirred, cooled to 0° C., and adjusted to pH 12-13 with 2 M aqueous NaOH solution. The mixture was transferred into a 2-L separating funnel and extracted with CH₂Cl₂ (2×1 L). The combined organic phases were washed with 1 M aqueous NaOH solution, dried over anhydrous Na₂SO₄, and evaporated. The residue was purified by flash column chromatography using CH₂Cl₂-MeOH (30:1) then CH₂Cl₂-MeOH (10:1) as the eluent to produce 57 g of (±)-N-((1-benzylpiperidin-3-yl)methyl)-N-(2-methoxyethyl)naphthalene-2-sulfonamide as a colorless oil.

Product appearance: colorless oil

Yield: 78%

TLC: R_{f}=0.57 (CH₂Cl₂-MeOH=10:1, v/v)

IR (ATR): 2928, 2803, 1452, 1333, 1154, 1115, 1072, 983, 883, 859, 817, 732, 699, 650, 615 cm⁻¹.

¹H NMR (400 MHz, CDCl₃): δ=0.95-1.04 (1H, m), 1.54 (1 H, bs), 1.66-1.74 (3H, m), 1.98 (2 H, bs), 2.76 (2H, d, J=30.4 Hz), 3.06-3.16 (2H, m), 3.21 (3 H, s), 3.30 (2H, t, J=6.3 Hz), 3.45-3.48 (4H, m), 7.23-7.30 (5H, m), 7.59-7.66 (2H, m), 7.76 (1 H, dd, J1=8.6 Hz, J2=1.8 Hz), 7.89-7.97 (3H, m), 8.38 (1H, d, J=1.4 Hz).

¹³C NMR (100 MHz, CDCl₃): δ=24.54, 28.21, 34.82, 48.04, 53.21, 53.89, 57.60, 58.66, 63.44, 71.10, 122.52, 126.85, 127.39, 127.78, 128.05, 128.36, 128.56, 129.07, 129.11, 129.14, 132.07, 134.59, 136.40, 138.25.

HRMS (ESI+): m/z calculated for C₂₆H₃₃N₂O₃S 453.2212; found 453.2209.

### Example 4: Synthesis of (±)-N-((1-Benzylpiperidin-3-Yl)Methyl)-N-(2-Methoxyethyl)Naphthalene-2-Sulfonamide Hydrochloride

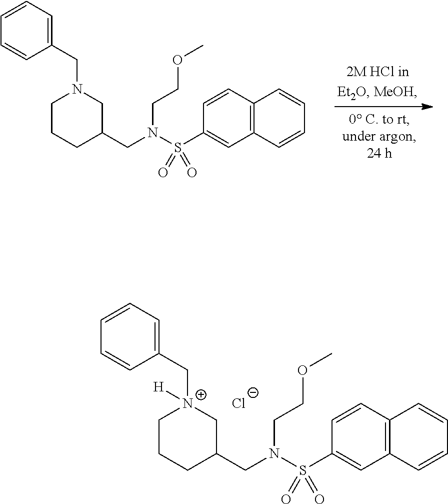 [SMILES: COCCN(CC1CCCN(Cc2ccccc2)C1)S(=O)(=O)c1ccc2ccccc2c1 ; COCCN(CC1CCC[NH+](Cc2ccccc2)C1)S(=O)(=O)c1ccc2ccccc2c1 ; [Cl-]]

To a 1-L round-bottomed flask containing (±)-N-((1-benzylpiperidin-3-yl)methyl)-N-(2-methoxyethyl)naphthalene-2-sulfonamide (57 g, 0.126 mol, 1.0 equiv), MeOH (300 mL) was added. A stirring bar was added to the resulting solution which was then stirred and agitated with a stream of argon for 30 min, and cooled to 0° C. 2 M HCl solution in Et₂O (69 mL, 0.139 mol, 1.1 equiv) was added with a double-tipped needle. The reaction mixture was allowed to warm up to room temperature, stirred for 24 hours and evaporated. Et₂O (500 mL) was added to the oily residue, and the flask was placed in an ultrasonic bath for 30 min. During this time, the oily residue transformed into a white solid. A stirring bar was added and the suspension was stirred for 12 hours at room temperature. The white precipitate was then collected in a Buchner funnel under suction filtration, washed with Et₂O (2×500 mL) and dried in a desiccator in vacuo at room temperature in the presence of crushed NaOH to constant mass to produce 49 g of (±)-N-((1-benzylpiperidin-3-yl)methyl)-N-(2-methoxyethyl)naphthalene-2-sulfonamide hydrochloride.

Product appearance: white solid

Yield: 79%

### Example 5: Biological Evaluation

I. Determination of in vitro ChE inhibition

1. Reagents and Procedure

The inhibitory potencies against huBChE and murine AChE (mAChE) were determined for all of these synthesized compounds using the method of Ellman (Ellman G. L. et al. Biochem. Pharmacol. 1961, 7, 88-95). 5,5-Dithiobis (2-nitrobenzoic acid) (Ellman's reagent; DTNB), and the butyrylthiocholine and acetylthiocholine iodides were purchased from Sigma-Aldrich (Steinheim, Germany). mAChE and recombinant huBChE at the stock concentration of 4.6 mg/mL in 10 mM MES buffer (pH 6.5) were used. The enzyme solutions were prepared by dilution of the concentrated stocks in phosphate-buffered solution (0.1 M, pH 8.0). The reactions were carried out in a final volume of 300 μL of 0.1 M phosphate-buffered solution, pH 8.0, containing 333 μM DTNB, 5×10-4 M butyrylthiocholine/acetylthiocholine and 1×10⁻⁹ M or 5×10⁻¹¹ M huBChE or mAChE, respectively. The reactions were started by addition of the substrate, at room temperature. The final content of the organic solvent (DMSO) was always 1%. The formation of the yellow 5-thio-2-nitrobenzoate anion as a result of the reaction of DTNB with the thiocholines was monitored for 1 min as the change in absorbance at 412 nm, using a 96-well microplate reader (Synergy™ H4; BioTek Instruments, Inc., USA). To determine the blank value (b), phosphate-buffered solution replaced the enzyme solution. The initial velocity (v0) was calculated from the slope of the linear trend obtained, with each measurement carried out in triplicate. For the first inhibitory screening, stock solutions of the test compounds (1 mM) were prepared in DMSO. The compounds were added to each well at a final concentration of 10 μM. The reactions were started by addition of the substrate to the enzyme and inhibitor that had been preincubated for 300 s, to allow complete equilibration of the enzyme-inhibitor complexes. The initial velocities in the presence of the test compounds (vi) were calculated. The inhibitory potencies are expressed as the residual activities (RA=(vi−b)/(vo−b)). For the IC₅₀ measurements, eight different concentrations of each compound were used to obtain enzyme activities of between 5% and 90%. The IC₅₀ values were obtained by plotting the residual enzyme activities against the applied inhibitor concentrations, with the experimental data fitted to Equation (1):

Y=Bottom+(Top−Bottom)/(1+10\hat{ }((LogIC₅₀−X)×HillSlope)) (1),

where X is the logarithm of the inhibitor concentration, and Y is the residual activity. For the fitting procedure, the Gnuplot software and an in-house python script were used.

2. Results

TABLE 1
Inhibitory activity of the compound with formula (I).

| | IC₅₀ + STD | IC₅₀ + STD |
|---|---|---|
| | (μM) or | (μM) or |
| | % inhibition at | % inhibition at |
| | 10 μM | 10 μM |
| Structure | huBChE | mAChE |
| 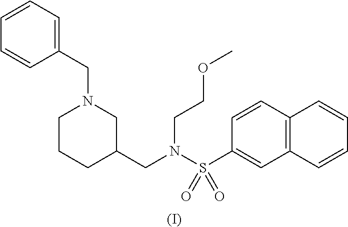 | 0.0049 ± 0.0003 μM | n.i. |

STD = standard deviation
n.i. = no inhibiton
huBChE = human butyrycholinesterase
mAChE = murine acetylcholinesterase

II. Determination of Cytotoxicity and Neuroprotective Effects

1. Cells and Reagents

The HepG2 cell line was obtained from American Type Culture Collection (LGC Standards, UK) and was cultured in RPMI 1640 medium (Sigma-Aldrich, St. Louis/MO, USA) supplemented with 10% fetal bovine serum (Gibco, Grand Island/NY, USA), 2 mM L-glutamine, 100 U/mL penicillin and 100 μg/mL streptomycin (all from Sigma-Aldrich) in a humidified chamber at 37° C. and 5% CO₂. (±)-N-((1-benzylpiperidin-3-yl)methyl)-N-(2-methoxyethyl)naphthalene-2-sulfonamide hydrochloride was used at 0.625 μM to 125 μM, in DMSO.

Human neuroblastoma SH—SY5Y cells were obtained from American Type Culture Collection (CRL-2266, Manassas, VA, USA). They were grown in Dulbecco's modified Eagle's medium (Sigma, St. Louis, MO) supplemented with 10% fetal bovine serum (HyClone, Logan, UT, USA), 2 mM L-glutamine, 50 U/mL penicillin and 50 μg/mL streptomycin (Sigma, St. Louis, MO, USA), in a humidified atmosphere of 95% air and 5% CO₂ at 37° C., and grown to 80% confluence. Prior to cell treatments, complete medium was replaced with reduced-serum medium (i.e., with 2% fetal bovine serum). (±)-N-((1-benzylpiperidin-3-yl)methyl)-N-(2-methoxyethyl)naphthalene-2-sulfonamide hydrochloride was prepared as a stock solution of 10 mM in DMSO and was used at concentrations of 1 μM to 100 μM. For the cytotoxic stimuli, Aβ₍₁₋₄₂₎ was dissolved in DMSO to give a 1 mM stock solution and 24 h prior cell treatment, the peptide was incubated at final concentration of 5 μM in reduced-serum medium in the absence and presence of (±)-N-((1-benzylpiperidin-3-yl)methyl)-N-(2-methoxyethyl)naphthalene-2-sulfonamide (1-10 μM) at 37° C., to induce Aβ aggregation.

2. Procedures

2.1. Metabolic Activity Assay

HepG2 cells (1×10⁴ cells/well) were treated for 24 h with the appropriate concentrations of the compounds of interest or the corresponding vehicle (control cells), in triplicates in 96-well plates. The metabolic activities were determined using the CellTiter 96 Aqueous One Solution Cell Proliferation Assay (Promega, WI/USA), according to the manufacturer instructions.

2.2. Cell Viability Assay

SH—SY5Y cells were seeded in 96-well plates (2×10⁴/well) and assessed in the MTS ([3-(4,5-dimethylthiazol-2-yl)-5-(3-carboxymethoxyphenyl)-2-(4-sulfophenyl)-2H-tetrazolium, inner salt) assay for their response to treatments with (±)-N-((1-benzylpiperidin-3-yl)methyl)-N-(2-methoxyethyl)naphthalene-2-sulfonamide hydrochloride. The cells were treated as described above, and cell viability was assessed after 48 h using the CellTiter 96 Aqueous One Solution Cell Proliferation Assay (Promega, Madison, WI, USA), according to the manufacturer instructions. Absorbance was measured with an automatic microplate reader (Tecan Safire2, Switzerland) at a wavelength of 492 nm. The data are presented as percentages of the control (DMSO).

2.3. Assessment of Cytotoxicity

The neuroprotective effect of (±)-N-((1-benzylpiperidin-3-yl)methyl)-N-(2-methoxyethyl)naphthalene-2-sulfonamide on cytotoxic effect of Aβ(1-42) was assessed by flow cytometry analysis using propidium iodide (PI; Sigma, St. Louis, MO, USA). PI does not cross cell membrane but stains DNA in cells when cell membrane is disintegrated. SH—SY5Y cells were seeded into a 24-well culture plate (1.2×10⁵/well) and next day, treated as described above. After 48 h treatment, cells were washed with pre-warmed PBS and further stained with PI solution (30 μM) for 15 min at 37° C. Cells were then analyzed for cytotoxicity by flow cytometry on FACS Calibur (BD Bioscience, San Jose, CA, USA). The percentage of PI positive cells (PI^{pos}) was evaluated using FlowJo software (Ashland, OR, USA) and results are presented as a relative fold increase of PI^{pos} corresponding vehicle treatment (DMSO control).

3. Results

3.1. Cytotoxicity

The LD₅₀ value for (±)-N-((1-benzylpiperidin-3-yl)methyl)-N-(2-methoxyethyl)naphthalene-2-sulfonamide with HepG2 cells was 58.67 μM, and with SH—SY5Y cells, 31.40 μM. These LD₅₀ values were almost 12,000-fold and >6,400-fold greater, respectively, than the concentrations needed to achieve 50% in vitro inhibition of huBChE.

3.2. Neuroprotective Effect

Treatment of SH—SY5Y cells with 5 μM Aβ₍₁₋₄₂₎ caused significant toxicity, whereas a clear dose-response neuroprotective effect was observed when the cells were exposed to Aβ(1-42) in the presence of (±)-N-((1-benzylpiperidin-3-yl)methyl)-N-(2-methoxyethyl)naphthalene-2-sulfonamide. Indeed, at 10 μM, this BChE inhibitor reversed Aβ₍₁₋₄₂₎-induced cell death.

III. Determination of In Vitro Pharmacokinetics

1. Reagents and Procedures

1.1. In Vitro Permeability Assay with Caco2 Cells

Caco-2 cells were obtained from American Tissue Culture Collection (ATCC) HTB.37, lot 61777387 and were used in reported experiments within one year. They were grown on Transwell® culture inserts with a polycarbonate membrane (diameter, 12 mm; pore size, 0.4 μm). 50,000 cells/filter membranes were used for seeding and the medium was changed every two days. At day 18, transepithelial electrical resistance (TEER) was measured for each filter with Caco-2 cell monolayers. If the TEER values were in the range of 450-750×cm², the Caco-2 cell monolayers were used for the subsequent testing of permeability at day 21. ABCB1 activity was confirmed with Rhodamine123 (Sigma Aldrich, Germany) a marker substrate of ABCB1, which had an efflux ratio of 7.3 (basolateral-to-apical apparent permeability coefficient was 7.7×10⁻⁶ cm/s and apical-to-basolateral apparent permeability coefficient was 1.1×10⁻⁶ cm/s). After inhibition with a selective ABCB1 inhibitor PSC833 (Tocris bioscience, UK) the efflux ratio was reduced to 2.5 (basolateral-to-apical apparent permeability coefficient was 2.5×10⁻⁶ cm/s and apical-to-basolateral apparent permeability coefficient was 1.0×10⁻⁶ cm/s). Fluorescence measurements with excitation at 485 nm and emission at 520 nm were used to quantify Rhodamine123 in samples taken as described later for (±)-N-((1-benzylpiperidin-3-yl)methyl)-N-(2-methoxyethyl)naphthalene-2-sulfonamide. The samples were acidified by dilution with the same volume of 0.01 M HCl before fluorescence measurements.

Ringer buffer with 10 mM d-glucose or 10 mM mannitol on apical and basolateral side of the tissue, respectively, was used as an incubation saline. The tissue was kept at 37° C. in a carbogen (95% 02 and 5% CO₂) atmosphere during the experiment. The experiment started by the addition of stock solutions of (±)-N-((1-benzylpiperidin-3-yl)methyl)-N-(2-methoxyethyl)naphthalene-2-sulfonamide hydrochloride and fluorescein in the donor compartment to provide final 100 μM donor concentration of (±)-N-((1-benzylpiperidin-3-yl)methyl)-N-(2-methoxyethyl)naphthalene-2-sulfonamide and 20 μM fluorescein. The stock solutions were added either to the apical or to the basolateral compartment to obtain bidirectional permeability measurements. Six samples were withdrawn in 20 min intervals. Each time 300 μl was taken from the 1500 μL basolateral acceptor compartment and 100 μL from the 500 μL apical acceptor compartment. These volumes were immediately replaced by the appropriate fresh incubation saline. The concentrations of (±)-N-((1-benzylpiperidin-3-yl)methyl)-N-(2-methoxyethyl)naphthalene-2-sulfonamide in the samples were determined by HPLC-UV immediately after the experiment. The analysis was performed on an Agilent 1100 system (degasser, binary pump, well-plate sampler, column thermostate and a diode-array detector) using a Zorbax Eclipse XDB-C18 column (4.6×75 mm, 3.5 μm) at 55° C. and a mobile phase consisting of 63% diluted phosphoric acid at pH 2.5 and 37% acetonitrile with a flow of 2.0 mL/min. The detection wavelength was 231 nm and the retention time was 1.38 min. All apparent permeability coefficients were calculated as described previously (Žakelj S. et al. Curr. Drug Metab. 2013, 14, 21-27).

1.2. Plasma Protein Binding

The equilibrium dialysis technique was used to separate the fraction of the bound to human plasma protein. The assay was performed in a 96-well format in a dialysis block constructed from Teflon to minimize the unspecific binding (Banker M. J. et al. J. Pharm. Sci. 2003, 92, 967-974). Plasma from human was used as the default protein containing matrix. Shortly, the protein matrix was spiked with (±)-N-((1-benzylpiperidin-3-yl)methyl)-N-(2-methoxyethyl)naphthalene-2-sulfonamide at 10 μM (n=2), with a final DMSO concentration of 1%. Acebutolol, quinidine and warfarin were tested in each assay as reference compounds, which yield protein binding that represent low, medium and high bindings to human plasma proteins, respectively. The dialysate compartment was loaded with the phosphate buffered saline (PBS, pH 7.4), and the sample side was loaded with equal volume of the spiked protein matrix. The dialysis plate was then sealed and incubated at 37° C. for 4 h. A control sample (n=2) was prepared from the spiked protein matrix in the same manner as the assay sample (without dialysis). The control sample served as the basis for the recovery determination. After the incubation, samples were taken from each compartment, diluted with phosphate buffer followed by addition of acetonitrile and centrifugation. The supernatants were then used for HPLC-MS/MS analysis using selected reaction monitoring. The HPLC conditions consisted of a binary LC pump with autosampler, a C18 column (2×20 mm), and gradient elution. The peak areas of (±)-N-((1-benzylpiperidin-3-yl)methyl)-N-(2-methoxyethyl)naphthalene-2-sulfonamide in the buffer and test samples were used to calculate percent binding and recovery according to the following equations:

Protein binding (%)=(Areaₚ−Area_{b})/Areaₚ×100 (3),

Recovery (%)=(Areaₚ−Area_{b})/Area_{c}×100 (4),

Areaₚ=peak area of the analyte in the protein matrix

Area_{b}=peak area of the analyte in the assay buffer

Area_{c}=peak area of the analyte in the control sample

The recovery determination serves as an indicator of reliability of the calculated protein binding value. Low recovery would indicate that the test compound is lost during the course of the assay, most likely due to non-specific binding or degradation.

1.3. Plasma Stability

The stability of (±)-N-((1-benzylpiperidin-3-yl)methyl)-N-(2-methoxyethyl)naphthalene-2-sulfonamide in human plasma was determined in a 96-well plate format (Di L. et al. Int. J. Pharm. 2005, 297, 110-119) Human plasma was pre-warmed at 37° C. water bath for 5 min, followed by addition of (±)-N-((1-benzylpiperidin-3-yl)methyl)-N-(2-methoxyethyl)naphthalene-2-sulfonamide hydrochloride at 1 μM with a final DMSO concentration of 0.5%. Propoxycaine and propantheline were tested as reference compounds simultaneously in each assay. The incubation was performed in a 37° C. water bath for 2 h. An aliquot of the incubation mixture was transferred to acetonitrile at 0, 0.5, 1, 1.5 and 2 h, respectively. Samples were then mixed and centrifuged. Supernatants were used for HPLC-MS/MS analysis using selected reaction monitoring, and peak areas were recorded for each analyte. The HPLC conditions consisted of a binary LC pump with autosampler, a C18 column (2×20 mm), and gradient elution. The area of precursor compound remaining after each time points relative to the amount remaining at time zero, expressed as percent, is calculated. Subsequently, the half-life (t_{1/2}) was estimated from the slope of the initial linear range of the logarithmic curve of the compound remaining (%) versus time, assuming first order kinetics.

1.4. Metabolic Stability

The metabolic stability, expressed as the intrinsic clearance (Clᵢₙₜ) of (±)-N-((1-benzylpiperidin-3-yl)methyl)-N-(2-methoxyethyl)naphthalene-2-sulfonamide using human cryopreserved hepatocytes, was determined in a 96-well plate format (Obach R. S. et al. J. Pharmacol. Exp. Ther. 1997, 283, 46-58). Cryopreserved hepatocytes (mixed gender and pool of 10 or more) were thawed, washed, and resuspended in Krebs-Heinslet buffer (pH 7.3). The reaction was initiated by adding test (±)-N-((1-benzylpiperidin-3-yl)methyl)-N-(2-methoxyethyl)naphthalene-2-sulfonamide at 1 μM (with a final DMSO concentration of 0.01%) into cell suspension (final cell density of 0.7 million viable cells per mL), and incubated for 0, 0.5, 1, 1.5 and 2 h, respectively, at 37° C./5% CO₂. The reaction was stopped by adding acetonitrile into the incubation mixture. Samples were then mixed, transferred completely to another 96-well plate, and centrifuged. Supernatants were used for HPLC-MS/MS analysis. As reference substances, four compounds were tested in each assay. Propranolol is relatively stable, whereas flurazepam, naloxone and 7-hydroxy-4-trifluoromethylcoumarin (HFC) are readily metabolized in human hepatocytes. In the HPLC-MS/MS analysis peak areas corresponding to the test compounds were recorded. Metabolic stability, expressed as percentage of the parent compound remaining, was calculated by comparing the peak area of the compound at the time point relative to that at time 0. The half-life (t_{1/2}) was estimated from the slope of the initial linear range of the logarithmic curve of the compound remaining (%) versus time, assuming first order kinetics. The apparent intrinsic clearance (Clᵢₙₜ in μL/min/million cells) was calculated from the half-life according to the following formula:

Clᵢₙₜ=0.693/(t_{1/2}×million cells/μL) (5).

1.5. Metabolite Identification

Principal Phase I (oxidative) and Phase II (conjugative) metabolites have been detected and characterized following previously described procedure (Obach R. S. et al. J. Pharmacol. Exp. Ther. 1997, 283, 46-58). Briefly, the test compound 2 (10 μL) was incubated at 37° C. in cryopreserved human hepatocytes (0.7 million cells/mL) for 120 min. Samples were taken at time 0 and the end of the incubation (2 h), respectively. A control sample (no test compound added) was also prepared and incubated for the same period of time. After the reaction was stopped by addition of acetonitrile, the supernatants were subjected to HPLC-MS analysis. Separation of analytes was achieved using Acquity HSS T3 column (100×2.1 mm, 1.8 μm, Waters). The sample solutions-supernatants (5 μL) were injected and eluted at a flow rate of 0.5 mL min-1, using a linear gradient of mobile phase A (0.1% formic acid in water) and mobile phase B (0.1% formic acid in MeCN). The gradient for mobile phase B was: 0-1.0 min, 5%; 1.0-7.0 min, 5%-80%; 7.0-7.5 min, 80%; 7.5-8.0 min, 80%-5%; 8.0-10.0 min, 5%. The HPLC was interfaced to a Waters Xevo G2 QT of system using electrospray ionization (source temperature: 150° C., desolvation temperature: 400° C., desolvation gas 400 L/h; capillary: 0.75 kV; sampling cone voltage 50V; extraction cone voltage: 2.7 V). A full-scan analysis in positive mode was performed over a mass-to-charge range suitable to cover the expected Phase I and Phase II metabolic product of the test compound (200-800 Da). An MSE acquisition method was used, which utilizes a high and low collision energy function such that precursor (MS) and product ion (MS/MS) information on all analytes in the sample is collected in a single analysis. Full-scan Total Ion Chromatograms (TIC) obtained from the time 0 and time 2 h sample, and also a blank matrix sample, was compared using the MetaboLynx software package of the MS system. With the application the sample data files were processed in an automated fashion to look for expected and unexpected metabolic products, and unique peaks in the incubation samples were directly compared to the control sample. Retention times and the respective MS and MS/MS spectra of the species detected in the incubated sample and not in time-zero or blank sample were identified and recorded.

2. Results

2.1. In Vitro Permeability Assay with Caco2 Cells

The permeability values (Pₐₚₚ) of (±)-N-((1-benzylpiperidin-3-yl)methyl)-N-(2-methoxyethyl)naphthalene-2-sulfonamide was determined in both directions, in eliminatory [basolateral-to-apical (B-A)] and absorptive [apical-to-basolateral (A-B)], and the efflux ratio [P_{app(B-A)}/P_{app(A-B)}] was calculated. The P_{app(B-A)} of (±)-N-((1-benzylpiperidin-3-yl)methyl)-N-(2-methoxyethyl)naphthalene-2-sulfonamide (29.1±2.9)×10⁻⁶ cm/s, was “high” according to the biopharmaceutical classification system of drug permeability since it is comparable to those of antipyrine, naproxen, propranolol and theophylline previously measured in the same experimental setting in the same laboratory (Žakelj S. et al. Curr. Drug Metab. 2013, 14, 21-27). In the opposite direction the P_{app(A-B)} was (30.0±10.7)×10⁻⁶ cm/s which results in an efflux ratio (P_{B-A}/P_{A-B}) of 0.97 and demonstrates that (±)-N-((1-benzylpiperidin-3-yl)methyl)-N-(2-methoxyethyl)naphthalene-2-sulfonamide is not subject to any significant active efflux mechanisms (Di, L. & Kerns, E. H. Transporter Methods. In Drug-Like Properties: Concepts, Structure, Design, and Methods from ADME to Toxicity Optimization 2nd edn Ch. 27, 339-350 (Academic Press, 2016).

2.2. Plasma Protein Binding

Equilibrium dialysis showed that (±)-N-((1-benzylpiperidin-3-yl)methyl)-N-(2-methoxyethyl)naphthalene-2-sulfonamide is 96% protein bound, which is higher than the threshold of 90%, above which a compound is classified as highly protein bound (Fesce R. and Fumagalli G. Drug Distribution and Ellimination. In General and Molecular Pharmacology: Principles of Drug Action 1st edn Ch. 5, (eds Clementi F. and Fumagalli G.) 45-60 (Wiley, 2015).

2.3. Plasma Stability

The in vitro half-life of (±)-N-((1-benzylpiperidin-3-yl)methyl)-N-(2-methoxyethyl)naphthalene-2-sulfonamide in human plasma was >120 min.

2.4. Metabolic Stability

The in vitro half-life of (±)-N-((1-benzylpiperidin-3-yl)methyl)-N-(2-methoxyethyl)naphthalene-2-sulfonamide in cryoperserved hepatocytes was 54 min. This half-life of (±)-N-((1-benzylpiperidin-3-yl)methyl)-N-(2-methoxyethyl)naphthalene-2-sulfonamide is comparable to other high extraction ratio reference compounds cleared by CYP enzymes that were used in the metabolic stability experiment as positive controls (Supplementary Table 7). The hepatocyte clearance of (±)-N-((1-benzylpiperidin-3-yl)methyl)-N-(2-methoxyethyl)naphthalene-2-sulfonamide was 18.2 μL/min/million cells.

2.5. Metabolite Identification

Three possible metabolites of (±)-N-((1-benzylpiperidin-3-yl)methyl)-N-(2-methoxyethyl)naphthalene-2-sulfonamide are N-debenzylated, demethylated, and a demethylated+glucoronide products.

IV. In Vivo Activity

Behavioral Testing in Dogs

1. Dog Recruitment

Thirty-one dogs older than 10 years were screened for the study. Of these, 14 were excluded from the study because of different abnormal findings observed during the initial examination, including 3 dogs with brain tumor confirmed with the computed tomography (CT); 2 dogs with markedly elevated alanine aminotransferase (ALT) and serum alkaline phosphatase (ALP); 3 dogs with renal disease; 3 dogs with marked hearing and visual deficit; 2 dogs with both renal disease and moderately elevated ALT and ALP and 1 dog with eclampsia.

After the owner's consent, dogs were subjected to physical and neurological examination including body weight and body condition score, blood results for complete blood count (CBC), clinical chemistry, urinanalysis, the canine dementia scale (CADES) questionnaire, and two clinical cognitive tests [food searching test (FST) and problem solving test (PST)]. After these thorough examinations, the decision was made whether the dogs were included in the study. Dogs with systemic illnesses that could interfere with their cognitive function were excluded from the study.

Only dogs aged 10 years or older were included in the study. According to the CADES score scale, all of them displayed at least mild cognitive impairment and were affected in at least two domains on the CADES score questionnaire. Dogs were not treated with any other medications for cognitive impairment. However, dogs that were included in the control group were allowed to be feed with the commercial therapeutic diet for cognitive decline, if they were on it at least 8 weeks before entering the study and if they maintained the same dietary protocol for the duration of the study. Dogs in the treatment group that were on a diet meant to treat cognitive decline were required to be off the diet for at least 30 days before enrollment and during the duration of the study.

2. Study Design

The study design was following the guidelines of the European Directive 2010/63/EU. Before starting the experimental therapy with their pets, each owner was required to sign an informed consent form. According to The Administration of the Republic of Slovenian for Food Safety, Veterinary and Plant Protection, no ethical permission was needed as the study was performed on clinical patients with the owners' consents. In total, seventeen dogs showing signs of cognitive dysfunction were included in the study. All eligible dogs were older than 10 years of age and exhibited signs associated with CCD. Upon enrollment, dogs were physically and neurologically examined, blood and urine analysis were performed. Additionally, a questionnaire based on a modified CADES scale score and two clinical tests (FST and problem PST) for assessing their cognitive function were performed. Dogs were randomly allocated to one of the following two groups: Control group (n=7) without treatment and the treatment group (n=10). Questionnaire and cognitive tests were performed every 2 months on all the dogs included in the study. Each treated dog received (±)-N-((1-benzylpiperidin-3-yl)methyl)-N-(2-methoxyethyl)naphthalene-2-sulfonamide hydrochloride for at least 6 months.

3. Physical and Neurological Examinations

Owners were observing their dog's health status daily during the duration of the study. In case of any serious or abnormal observations, they had to contact the participating veterinarian. Physical and excessive neurological examinations (behavior and consciousness, cranial nerves, gait and posture, postural reactions, spinal reflexes) were carried out by the veterinarian and included an assessment of all body systems. A full spectrum of neurobehavioral symptoms including apathy, anxiety, staring blankly, confusion or aimless walking, vocalization during the night, aggression, signs of compulsive and stereotyped behavior, etc. were obtained by questioning the pet owners. Dogs were monitored during examinations. Their responses to familiar and unfamiliar people and objects were observed. Exams were performed at the beginning of the study and every two weeks for the first two months if dogs were included in the treatment group. Thereafter, exams were performed every two months for the duration of the study (at least for 6 months). The control group was examined only every 2 months until the end of the study (for at least 6 months).

4. Behavior Examination

Changes in behavior of all dogs were evaluated by the same veterinarian. Each check-up included observations of geriatric dogs and the collection of information provided by pet owners. Thorough conversations with the owners were performed to identify indicators of any neurological or metabolic diseases that would lead to rejecting CCD as the main diagnosis. Questions focused on information about appetite, drinking, loss of perception (attempts to pass through narrow spaces or the wrong side of the door), disorientation, nigh-time waking, anxiety, excessive vocalization, house soiling, aggression, changes in activity, attention-seeking behavior, aimless behavior (stargazing, circling, stereotyped walking), panting, muscle tremors/shaking, memory loss, and other sings that were noticed by owners. An important consideration was also the onset and progression of clinical signs. To quantify the cognitive decline in dogs, we used a modified scoring system CADES, adapted and modified from Osella M. C. et al. Appl. Anim. Behav. Sci. 2007, 105, 297-310, Salvin H. E. et al. Vet. J. 2011, 188, 331-336, and Madari A. et al. Appl. Anim. Behav. Sci. 2015, 171, 138-145 (Table 2). The questionnaire was filled out by the veterinarian through an interview with the dog's owner. The questionnaire is divided into four domains according to different ability areas. Domain A is reporting the spatial orientation, domain B the social interactions, domain C house soiling, and domain D sleep-wake cycles (Table 2). After completion and scoring of the questionnaires, canine patients were classified into four stages (mild, moderate, severe, and extreme cognitive impairment). The classification was based on the severity of the changes in behavior, their frequency, and the number of affected domains.

5. Cognitive Tests

FST and PST were performed on each dog included in the study. The design of these tests was based on previously published tests for canine cognitive dysfunction (González-Martinez A. et al. Vet. J. 2013, 198, 176-181). The cognitive tests were evaluated by the same veterinarian. Dogs were tested in the morning after at least 10 hours of the fasting period.

5.1. Food Searching Test (FST)

This test was based on testing the dog's ability to find hidden food. The dog was seated in the middle of the room while being on the leash. The veterinarian was positioned in front of the dog (60 cm away) and showed the dog a piece of its favorite treat. While maintaining visual contact with the dog and still communicating with the dog, showing the food, the veterinarian than moved backward and placed the food in the corner of the room. The veterinarian than stared at the food and pointed to it with his hand for 2-3 s to increase the dog's visual processing. The owner was then asked to leave the room with the dog and wait outside for 15 s. After returning to the room, the dog was placed into the center of the room unleashed and allowed to freely explore the room for 1 minute. No verbal or other clues were allowed. The procedure was repeated twice. In each run the test was scored as follows: the dog goes directly to the food (1 point), the dog searches for the food and finds it within 1 minute (2 points); the dog searches for the food but does not find it within 1 minute (3 points); the dog does not make any attempt to search for the food (4 points). The FST total score was the average of both runs.

5.2. Problem Solving Test (PST)

In this test, the ability of the dog to obtain the food by manipulating an object was assessed. The owner showed the dog its favorite food and the dog was allowed to sniff and lick it. After that, the food was placed on the floor in front of the dog and covered with a transparent plastic box that had been turned upside down. The dog was given 2 minutes to find a way to remove the box and get the food. The test was performed twice and scored as follows: the dog obtained the food within 2 minutes (1 point), the dog tried to obtain the food but did not obtain it within 2 minutes (2 points), the dog sniffed the box but did not try to get the food (3 points), the dog did not make any attempt to get the food (4 points). The PST total score was the average of both runs. The sum of FST and PST test scores was used for the scoring and the statistical analysis.

6. Blood Work and Urinalysis

Complete hematology with cytology was performed to investigate the presence of anemia (portosystemic shunt, hypothyroidism, lead poisoning, anemia of chronic disease . . . ), abnormal white blood cell count (inflammatory or neoplastic process) or abnormal platelet count (e.g. paraneoplastic thrombocytopenia, thrombocytosis associated with hyperadrenocorticism). Serum biochemistry was performed to evaluate renal (urea, creatinine) and liver parameters (ALT, ALP, AST), but also included the measurement of serum glucose, serum cholesterol, serum albumin, globulin, Na and Ca. Urinalysis was also performed. In dogs included in the treatment group, the blood work and urinalysis were performed every two weeks to make sure the drug did not affect their kidney and liver function.

7. Statistical Analysis

Results are presented as mean+SD. Age between groups was compared by one-way ANOVA. CADES score and results from behavioral tests were analyzed by repeated measures ANOVA with treatment as the independent variable and clinical test (before the treatment, 3 months, and 6 months after the treatment) as within factor. CCD state of the dogs was compared with X2 test separately for tests before the start of the treatment and at three months after treatment. Change in CADES score and behavioral tests score during the study was tested separately in the treated and untreated groups by one-way ANOVA with the time of clinical testing as an independent variable. Differences were considered statistically significant with p<0.05.

TABLE 2
Information on dogs' cognitive status
based on CADES score and their affected domains

| Cognitive state | Affected cognitive domains | | | |
|---|---|---|---|---|
| Moderate cognitive | Domain AB | Domain BC | Domain ABC | / |
| impairment | 4 (36.4%) | 5 (45.5%) | 2 (18.2%) | |
| Severe cognitive | Domain ABC | Domain ACD | Domain BCD | Domain ABCD |
| impairment | 1 (16.7%) | 1 (16.7%) | 2 (33.3%) | 2 (33.3%) |

There was no statistically significant difference in CADES score between treated and untreated groups at the beginning of the study (p>0.05). The average CADES score in untreated group was 43.7+/−16.1 (range: 24-65) and in treated group 39+/−16.4 (range: 19-65).

8. Results

8.1. Sample Characteristics

Dogs included in the study were of different breeds (small to middle-sized: six cross breeds, three Maltese dogs, two Tibetan Terriers, two Dachshunds, two Pugs, one Standard Schnauzer, one American Stafford, and one Bull Terrier). Female dogs slightly (64.7%) outnumbered male dogs (35.3%), but there was no statistically significant difference found for sex between treated and untreated groups.

The mean age of dogs was 14.4+/−1.3 years (range: 13-17 years). There was no statistically significant difference in age between treated (14.2+/−1.3 years) and untreated (14.8+/−1.3 years) groups. All females were spayed, but all males were intact.

8.2. Disease Severity and Staging

Among the 17 dogs that fulfilled criteria for cognitive impairment, 11 dogs had moderate cognitive impairment and 6 dogs severe cognitive impairment. Seven dogs with moderate cognitive impairment and three dogs with severe cognitive impairment were included in the treatment group. We had to stop treatment in all dogs with severe cognitive impairment, since they all showed signs of gastrointestinal problems with vomiting and diarrhea after treatment, even though they did not show signs of renal or liver disease before or after treatment. Dogs showing moderate cognitive impairment continued the treatment without any noticeable side effects for 6 months.

In dogs with moderate cognitive impairment, pet owners observed behavioral changes such as hyperactivity during the night time, anxiety, disorientation, and changes in altercations with people and other animals. The number of affected domains varied from 2 to 4. All animals showed an apparent decline in at least two domains over 6 months' period (Table 2). In dogs with severe cognitive impairment severe behavioral changes were reported by owners and all animals showed apparent decline over 6 months' period in at least three domains (Table 2).

8.3. Comparison of Follow-Up Results Between the Treated and Untreated Groups

8.3.1. CADES Score

Dogs included in the study underwent clinical examinations three and six months after the beginning of the treatment.

In the untreated group, there was a statistically significant decline in cognitive impairment after three months in all seven patients (p<0.001). Five out of seven dogs in the untreated group were euthanized between 3 to 5 months after inclusion in the study, the other two remained in the study until the end and further decline in cognitive functions was noticed. The average CADES score after three months in the untreated group was 64.0+/−12.8 and after six months the average CADES score of the two remaining dogs was 80.5. The difference between the initial examination and examination at 3 months was statistically significant with p<0.05)

The average CADES score in treated patients after three months was 17.1+/−10.6 and after six months was 12.6+/−3.0. The difference in CADES score between untreated and treated groups was significantly different at both 3 months and 6 months (p<0.001). The difference in CADES score was also significant at 3 and 6 months in comparison to the beginning of the study in the treated group (p<0.001) and at 3 months in comparison to the beginning of the study in the control group (p<0.05).

8.3.2. Number of Affected Domains on CADES Scale in Treated and Untreated Patients

To identify the phenotypic variability of the cognitive impairment in treated and untreated patients the number of dogs with selective impairment of domains was quantified. After three months 42.8% of untreated dogs had extreme cognitive impairment with all of the domains (A, B, C, and D) being affected. In 57.2% of untreated dogs' severe cognitive impairment was observed with at least three domains being affected (A, C, and D). After six months five out of seven dogs were euthanized since there was a huge increase in the severity of the cognitive impairment. In the remaining dogs, an extreme cognitive impairment according to CADES score with all four domains being affected (A, B, C, and D) was observed.

In the treated group improvement in cognitive abilities was observed and all dogs included stayed alive throughout the study. After three months only 14.3% of dogs showed severe cognitive impairment with three affected domains (A, C, and D). In others (85.7%) moderate cognitive impairment was noticed with only two affected domains. After six months all dogs showed moderate cognitive impairment with low CADES score. In all treated dogs improved memory, cognitive functions, and learning abilities were noticed. The only complaint owners observed were mostly reduced ability to do tasks previously learned and reduced ability to learn a new task. Some dogs still had problems with house soiling, but it happened less frequently. None of the dogs had problems during sleeping and they did not show signs of anxiety. All owners with treated dogs reported that the quality of life and dog-owner interaction drastically improved.

8.4. Problem Solving Tests

The test score results for food searching and problem solving tests in untreated and treated dogs are summarized in the Table 3.

TABLE 3
Problem solving test scores (sum of FST and
PST test) in dogs with cognitive impairment

| | Test results | | |
|---|---|---|---|
| Patient | At the | After three | After six |
| Number | beginning | months | months |
| Untreated group | | | |
| 1 | 8 | 8 | euthanasia |
| 2 | 4 | 8 | 8 |
| 3 | 2 | 8 | euthanasia |
| 4 | 8 | 8 | euthanasia |
| 5 | 3 | 5 | euthanasia |
| 6 | 6 | 8 | 8 |
| 7 | 8 | 8 | euthanasia |
| Treated group | | | |
| 1 | 4 | 4 | 2 |
| 2 | 5 | 4 | 3 |
| 3 | 6 | 2 | 2 |
| 4 | 4 | 2 | 2 |
| 5 | 7 | 4 | 4 |
| 6 | 5 | 4 | 4 |
| 7 | 4 | 2 | 2 |
| 8 | 4* | excluded | excluded |
| 9 | 6* | excluded | excluded |
| 10 | 8* | excluded | excluded |

8.4.1. Food Searching Test (FST)

At the beginning of the study, the average food searching score was 2.4+/−1.1 in the group of dogs with moderate cognitive impairment, and 3.5+/−0.8 in the group of dogs with severe cognitive impairment. The difference between groups was statistically significant (p<0.05). During the FST tests, 72.7% of dogs with moderate cognitive impairment searched for the dropped food. Among those 18.2% went directly towards the food and 45.5% were able to find it within a minute of searching. In a group of dogs with severe cognitive impairment, 33.3% were searching for the food, and only 16.7% were able to find it within one minute.

8.4.2. Problem Solving Test (PST)

At the beginning of the study, the average problem solving score was 2.3+/−1.0 in the group of dogs with moderate cognitive impairment, and 3.5+/−0.8 in the group of dogs with severe cognitive impairment. The difference between groups was significant (p<0.05). During the PST tests, 54.5% dogs with moderate cognitive impairment tried to obtain the food and half of those (27.3%) were able to obtain the food within a 2 min period. Among others, 36.6% of dogs sniffed the box but did not try to get the food and 9.1% did not make any attempt to get the food. In contrast, two-thirds of dogs with severe cognitive impairment did not make any attempt to get the food.

8.5. Comparison of Results for PST and FST Between the Treated and Untreated Groups

Dogs included in the study were followed up and problem solving tests were repeated three and six months after the inclusion.

At the beginning of the trial, there was no statistically significant difference in problem solving score between untreated (5.6+/−2.6) and treated group (5.3+/−1.4) (p>0.05).

After three months a decline in solving both tests were was noticed in the untreated group (7.6+/−1). Most dogs (85.7%) did not show any attempt to search for the food (FST) and they did not even sniff the box where the food was hidden (PST). Five out of seven dogs in the untreated group were euthanized between 3 to 5 months after inclusion in the study, the other two remained in the study until the end. In one of these dogs the problem solving score remained 8, but in the other further decline in problem solving was observed. However, an average score in the whole group was lower since euthanized dogs were not included in the statistics.

In contrast, dogs that were treated were able to solve the tests better after three months (3.1+/−1.1) and they performed even better after six months (2.7+/−1.0). The difference between treated and untreated groups was statistically significant at 3 months with p<0.001.

1. A compound of formula (I) 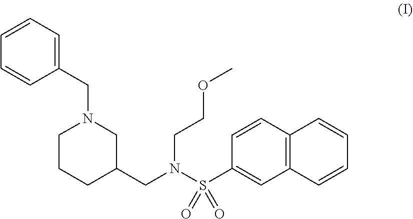 [SMILES: COCCN(CC1CCCN(Cc2ccccc2)C1)S(=O)(=O)c1ccc2ccccc2c1]
optionally in the form of a stereoisomer, such as enantiomer, or a mixture of at least two stereoisomers, such as at least two enantiomers, or a pharmaceutically acceptable salt, hydrate or solvate thereof, for use in the treatment of canine cognitive dysfunction or other form of dementia in dog.

2. Use of a compound of formula (I) 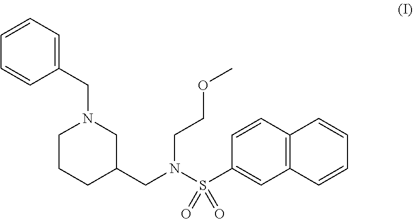 [SMILES: COCCN(CC1CCCN(Cc2ccccc2)C1)S(=O)(=O)c1ccc2ccccc2c1]
optionally in the form of a stereoisomer, such as enantiomer, or a mixture of at least two stereoisomers, such as at least two enantiomers, or a pharmaceutically acceptable salt, hydrate or solvate thereof, in the manufacture of a medicament for use in the treatment of canine cognitive dysfunction or other form of dementia in dog.

3. A pharmaceutical composition for use in the treatment of canine cognitive dysfunction or other form of dementia in dog, comprising a therapeutically effective a mound of formula (I) 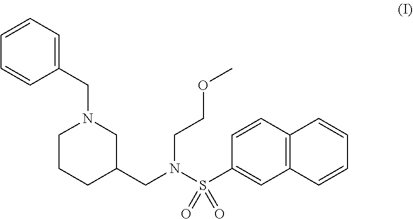 [SMILES: COCCN(CC1CCCN(Cc2ccccc2)C1)S(=O)(=O)c1ccc2ccccc2c1]
optionally in the form of a stereoisomer, such as enantiomer, or a mixture of at least two stereoisomers, such as at least two enantiomers, or a pharmaceutically acceptable salt, hydrate or solvate thereof.
